(19) United States
(12) Patent Application Publication
(10) Pub. No.: US 2022/0062010 A1
(43) Pub. Date: Mar. 3, 2022

(54) MEDICAL APPLIANCE FOR A JOINT OF A PERSON AND METHOD FOR OPERATING A MEDICAL APPLIANCE

(71) Applicant: MEDI GMBH & CO KG, Bayreuth (DE)

(72) Inventors: Marco NAUMANN, Pegnitz (DE); Hans-Peter LEHNER, Bindlach (DE)

(73) Assignee: MEDI GMBH & CO KG, Bayreuth (DE)

(21) Appl. No.: 17/415,637

(22) PCT Filed: Oct. 31, 2019
(86) PCT No.: PCT/EP2019/079838

§ 371 Date: Jun. 17, 2021

(30) Foreign Application Priority Data
Dec. 18, 2018 (EP) 18213421.3

(51) Int. Cl. A61F 2/68; A61F 5/01

(52) U.S. Cl. CPC A61F 2/68; A61F 2002/689; A61F 5/013; A61F 5/0123

(57) ABSTRACT

Medical appliance (9, 9a, 9b, 9c) for a joint (1) of a person (10), which has at least two parts (11) which are to be placed on the joint (1) and can be moved with respect to one another by the joint (1) and at least one detection device (13) for detecting a relative movement of the parts (11), wherein the medical appliance (9, 9a, 9b, 9c) comprises an evaluation device (16), having:
a determination unit (17) for determining, from detection data of the detection device (13), a range of movement (5) of the joint (1), which range of movement is actually used when the medical appliance (9, 9a, 9b, 9c) is worn,
a comparison unit (18) for comparing the range of movement (5) used with a desired range of movement (6), and
a support unit (19) for determining support information guiding the person (10) to the most extensive use possible of the desired range of movement (6), said support information being dependent on the result of the comparison.

The medical appliance (9, 9a, 9b, 9c) further has an output device (20) for outputting the support information to the person (10).

The invention relates to a medical appliance for a joint of a person, the appliance having at least two parts which are to be placed on the joint and can be moved with respect to one another by the joint and at least one detection device for detecting a relative movement of the parts. The invention further relates to a method for operating such a medical appliance.

Medical appliances for a wide variety of joints of a person have already been proposed in the prior art in a variety of ways and comprise, for example, bandages, orthoses, belt systems, stockings and other garments that act in particular compressively. Within the scope of this description, “medical” should also be understood to mean sports applications that are characterized by desired positive effects for the anatomy and/or health and/or performance that are based on medical considerations. Medical appliances of this type for joints thus also include sports bandages and the like, for example. Furthermore, within the scope of the present description, the spine should also be regarded as a joint or group of joints to which medical appliances can be applied.

The medical appliance may also exist independently of such treatment devices that may be formed by, or comprise, the aforementioned parts. For example, it can be explicitly designed to detect the joint movement as a relative movement of the parts, and may thus in particular form an auxiliary device or accessory for a treatment device, such as an orthosis, a bandage, a stocking or the like. In the case of medical treatment products, or even more specifically treatment devices, the two parts can be formed by separate components that are, for example, rigid and are connected to one another in an articulated manner. However, it is also possible for the parts to be formed as subregions of a single flexible object, for example a stocking.

Persons who use medical appliances that are aimed at a therapeutic treatment and/or at protecting the joint tend to adopt protective postures or carry out protective movements that may lead to disadvantageous effects during treatment with the medical appliance. If the appliance comprises, for example, an orthosis that limits the range of movement of the joint, for example of a knee, the person normally will not use the full range of movement permitted by the orthosis but will rather use a significantly smaller range of movement, which in its extent will be also significantly smaller than the range of movement desired by a therapist, for example. This can be determined by a detection device of the appliance which detects a relative movement of the parts.

For example, WO 2016/176544 A1 discloses a sensor and feedback platform for use in orthotic and prosthetic devices. Parameters measured by means of sensors, which can describe a movement, are supplied to data processing and/or data representation units for evaluating and/or performing actions. Specifically, it is proposed there to use as a sensor mechanism an inductive sensor that interacts with conductive material in a belt or strip in order to produce sensor data that indicates the location of the inductive sensor relative to the belt/strip.

Disadvantageously, however, it can be determined only subsequently via such a platform that the range of movement used did not correspond to a desired or even maximum permissible range of movement, in order to thereafter inform the person using the orthosis or prosthesis. Here, improvements in the utilization of the range of movement can be brought about only to a limited extent since there is no intuitively understandable connection between the evaluation and the previously performed movements for the person. The use of protective postures or protective movements, however, leads to deficits in muscle strength and/or the movement function when a medical appliance is worn, which is undesirable.

The object of the invention is, therefore, to provide an improved way of avoiding protective postures, in particular without having to dispense with any existing protective function of a treatment device.

According to the invention, this object is achieved by a medical appliance according to claim 1 and a method for operating a medical appliance according to claim 14. Advantageous embodiments result from the dependent claims.

According to the invention, a medical appliance of the type mentioned at the outset therefore has an evaluation device which comprises:
a determination unit for determining, from detection data of the detection device, a range of movement of the joint, which range of movement is actually used when the medical appliance is worn,
a comparison unit for comparing the range of movement used with a desired range of movement, and
a support unit for determining support information guiding the person to the most extensive use possible of the desired range of movement, said support information being dependent on the result of the comparison,
   wherein the medical appliance further has an output device for outputting the support information to the person.

In the context of the present invention, medically oriented sports aids should also be considered as medical appliances, as mentioned at the outset. The appliance can also be provided for the spine as a group of joints, so that in other words it is a medical appliance for a joint or a spine. Other joints in which such a medical appliance can be used are, for example, knee joints and/or elbow joints. The range of movement must also be parameterized in each case with respect to the respective joint. In the case of a single-axis joint, such as a knee, the range of movement can be defined by angles between which the movement takes place or is to take place. For example, a desired range of movement can thus be defined by a target angle in at least one direction of movement, for example flection and/or extension in a knee.

The medical appliance may be a standalone appliance, but is preferably at least assigned to, or comprises, a treatment device, in particular a treatment device formed by, or comprising, the parts. Treatment devices can then, for example, be bandages, orthoses, garments, in particular compression garments, belt systems and/or splint systems. In particular, it is thus conceivable for the appliance to be assigned, so to speak, to at least one treatment device as an auxiliary device, which will be discussed in more detail below.

A basic idea of the present invention is thus to track the current movement of the joint by means of the detection device and to compare it as promptly as possible with a desired range of movement specified, for example, via a setting device, in order to output from this, if necessary, a piece of support information which leads the person to the greatest possible use of the desired range of movement. In other words, by means of the medical appliance proposed according to the invention, the person will not only receive the Information that, for example, it has adopted a non-intended protective posture, but it will be at the same time guided to a desired, in particular a targeted, presettable range of movement, so that a type of pro-activity is given.

In particular, it can therefore be provided that—where support information is not generally to be always output—at least one information criterion is provided in the support unit, which at least evaluates the comparison result, and that upon its fulfillment one piece of or the support information is output. Here, different support information can be assigned to different information criteria and/or different support information can be determined, in particular by further evaluation of the comparison result, upon fulfillment of a specific information criterion.

Specifically, the information criterion may compare at least one degree of usage with a threshold value, for example to what extent the desired range of movement is utilized and/or for which portion of movement cycles of the joint it was sufficiently utilized, for which purpose a further threshold value may possibly be provided.

In other words, it may thus be said that, by evaluating the comparison result, the support unit provides the person with information in such a way when the degree of usage is too low that said person is led to use the movement possibilities such as to reach the desired range of movement. Preferably, a lack of usage information describing the specific nature of the lack of usage can be determined in this case from the comparison result and taken into account in the parameterization of the support information, especially possibly of the output time as well. A lack of usage determined, for example, in the case of a knee may be that the flection is too small, since too large a distance from the flection target angle, which limits the desired range of movement, is given. The parameterization of the support information as a function of a type of lack of usage advantageously communicates to the person where improvement potential exists. A preferred embodiment also provides that a current state of movement is also taken into account in the parameterization of the support information, especially for temporal coordination of the support information and/or for instructing the person to change the state of movement for better utilization of the desired range of movement.

In summary, based on the comparison result the support information is generally designed and chronologically set such that it provides information leading to a better utilization of the desired range of movement, in particular intuitively and in the movement situation itself.

The desired range of movement, which can preferably be set or adjusted by means of a setting device, thus ultimately corresponds to a target to which the person, for example a patient or athlete, is guided or led by a corresponding design of the support information. In particular, it is thus possible to guide a patient from a currently used range of movement corresponding to a protective posture or protective movement to a therapeutically useful, currently desired range of movement, which can in particular be individually predefined by the therapist.

As already explained, although the medical appliance may only be assigned to a treatment device, it preferably comprises the treatment device, it thus ultimately provides it with an additional functionality. Specifically, it can be provided for the appliance to comprise, in particular as a treatment device and/or implementing the parts, at least one stocking-like enclosure section which is to be pulled over the joint and/or wrapped around the joint as a bandage and/or designed as a piece of clothing and/or as a belt system and/or at least one articulated guide element, in particular at least two hinged, rigid articulated guide elements. A treatment device optionally formed in this way can thus in particular be or comprise a bandage, an orthosis, a stocking, a back splint for the spine, a garment and/or a belt system.

At this point, it should also be noted that in the event of assignment to a treatment device, the latter does not have to be permanent or fixed, but it is absolutely conceivable for example to use the medical appliance, which can in particular be detachably connected to a treatment device, throughout an entire therapy or the like, for example first with an orthosis as the treatment device, then with a bandage as the treatment device, then with a stocking as the treatment device, and finally with a belt or the like as the treatment device.

In specific cases, it may be provided for the detection device to comprise at least one sensor which is integrated into at least one part or is detachably or non-detachably attached or attachable to at least one part and/or to a body region moving with a part. At this point, it should be noted once again that the parts movable relative to one another do not necessarily have to be two separate components of the appliance or of a treatment device, but it is absolutely also conceivable to use a flexible component, wherein the parts are then formed by subregions of the component. Such flexible components may comprise, for example, the aforementioned stocking, bandage elements made of compression material, flexible splints, belts, and/or the like. In particular when the parts are implemented as part of an enclosure section, it may be expedient to already integrate sensors or sensor parts of the detection device into the corresponding component, for example into the enclosure section, or to deliberately provide means for preferably detachably fastening to the corresponding component, specifically the respective parts. Hook-and-loop fasteners, for example, can be used for detachable fastening, but also snaps and the like. This is particularly expedient if suitable positions for the sensors of the detection device are already defined by the parts or more specifically the treatment device and the manner in which they are applied, so that finally attachment can take place at defined positions on the corresponding parts via the fastening means.

In a further specific embodiment, the detection device can be designed to measure a distance between two sensors and/or sensor parts and/or a joint angle and/or at least one dynamic variable of the joint movement, in particular an angular velocity and/or an angular acceleration. If, for example, it is a uniaxial joint, the movement of the joint and thus of the parts relative to one another takes place as a pivot about this joint axis. Two sensors or two sensor parts of a sensor can measure the distance between the sensors or the sensor parts and thereby infer a joint angle or also dynamic variables over time. Of course, this can also be used in joints with multiple joint axes, for example in ball joints and the like. As regards the application of the medical appliance in a spine, more complex movement spaces may also be defined, in particular also such that jointly map the movement of a plurality of individual vertebral joints.

Expediently, the at least one sensor can have a measuring principle using magnetism and/or induction. Such sensors, as described for example in WO 2016/176544 A1 mentioned at the outset, are particularly suitable for sufficiently accurately measuring the movement of the parts relative to one another; however, other sensor principles are of course also conceivable, such as capacitive distance-measuring sensors and the like.

It is expedient to realize at least some of the components of the medical appliance, in particular the evaluation device, remote from the joint, i.e., in particular external to a treatment device. Parts of the detection device can also in principle be provided remote from the joint or a treatment device. While it is in principle conceivable to realize the communication between such spatially separately realized components of the medical appliance by wire, it is preferred within the scope of the present invention to use wireless communication, in particular radio communication, in order to impede the person as little as possible. For example, proprietary but also standardized communication methods can be used for wireless communication, such as Bluetooth or the like. Such wireless communication can take place, for example, between the evaluation device and the detection device and/or sub-components thereof.

However, it is of course also possible within the scope of the present invention to arrange the entire medical appliance, i.e. in particular both the detection device and the evaluation device and the output device, on the joint itself, in particular on or as part of a treatment device. In particular, compact embodiments are also possible, which then preferably have haptic and/or acoustic output devices.

The appliance can preferably have a setting device for setting the desired range of movement of the joint which the person is to use when wearing the appliance. While it is in principle preferred within the scope of the present invention to permit a manual setting of the desired range of movement, for example by a therapist or other user, optionally even by the person itself, it may also be expedient in many embodiments to preferably additionally, or alternatively, also allow automatic setting of a desired range of movement by the setting device. For example, a therapy progress and/or a predetermined therapy plan can be used here, optionally also determinable by the detection device itself.

In embodiment of the invention, the setting device can have an electronic and/or mechanical setting means, in particular one that exists independently of a treatment device formed by the parts, in particular a hand-held mobile device. This means that the setting device can be designed in many different ways within the scope of the invention. In particular when a corresponding setting means of the setting device is provided close to the joint, in particular on a treatment device, the setting process can also include a mechanical component, for example in that the at least one joint or at least the setting device is brought into a position delimiting the desired range of movement, which position can then be stored accordingly. However, it is also particularly advantageous to provide electronic setting means, in particular for manual and/or automatic setting of the desired range of movement, which setting means can also be implemented at least partially by a hand-held mobile device. The hand-held mobile device, for example a mobile phone, can have, for example, a user interface via which desired ranges of movement and/or criteria for their automatic determination can be entered. For this purpose, in the case of a smartphone and/or tablet, a corresponding computer program, i.e. an application (app), can, for example, be provided as a hand-held mobile device.

Especially with regard to such an embodiment, a particularly preferred development of the present invention provides for the mobile device to additionally comprise a computing device designed as at least one part of the evaluation device, on which computing device there is an application forming the mobile device as a setting means and as the part of the evaluation device. In one exemplary embodiment, it can thus be provided for the hand-held mobile device to communicate with the detection device, preferably wirelessly, and to evaluate the detection data of the detection device by means of the computing device, in particular in order to compare the range of movement used to a desired range of movement and to determine whether support information is to be output and/or how the support information must be parameterized in order to be able to lead the person to the greatest possible use of the desired range of movement. In this context, an output means of the hand-held mobile device can otherwise also serve as an output device, for example a speaker for the acoustic output of support information and/or a display designed in particular as a touchscreen can also be used for the optical output of support information. In this way, the equipment and/or the computing power of the hand-held mobile device is thus optimally utilized in order to provide only a few components of the medical appliance at the joint itself. For example, it is advantageous if only the detection device and possibly the treatment device are to be arranged on the joint itself.

Another embodiment, which can optionally be used as setting means in addition to a hand-held mobile device, provides for the setting device, in particular comprising at least one setting button adopting a current joint position as a limit, for example a maximum or minimum, of the desired range of movement, to be implemented on or as part of the treatment device and/or to be designed to evaluate detection data of the detection device to determine a current joint position as a limit, for example a maximum or minimum, of the desired range of movement. In a specific development, it is thus conceivable that, for example, the treatment device has a setting button, for example a set button, in order to use current joint positions to define the desired range of movement. In particular, detection data of the detection device can be used here in order to be able to identify the current joint position, wherein other embodiments are of course also conceivable. It should be noted that a control element corresponding to a set button can of course also be implemented on a user interface of a hand-held mobile device. In both embodiments, that is, both when the setting button is provided on the treatment device or on at least one part and/or when the setting button is realized via a user interface of the mobile device, a reset button may also be provided in addition to such a set button, which may be used, for example, during re-positioning of the medical appliance, specifically thus of at least the parts, on the patient in order to reset all settings and/or to display, for example, basic positions for calibrating the detection device.

It should be noted at this point, in particular also with regard to a manual setting via the setting device, that of course further adjustments can also be made via the setting device in a corresponding embodiment, in particular the setting of support parameters related to the support information. For example, in addition to specifying a desired range of movement, the described setting devices can also be used to define portions of the desired range of movement in which support is actually to take place (also cf. the information criterion discussed above), at which (also relative) points in time a support is to take place, what the general intensity of the support information is supposed to be, and the like. Furthermore, it may be provided that at least one further range parameter associated with the desired range of movement can also be set via the setting device, in particular a time parameter describing the time period over which the comparison is to be evaluated and/or a type of movement for which the desired range of movement is to be used.

As already mentioned, the setting device does not have to (exclusively) be suitable for manually setting the desired range of movement but can also implement an at least partially automatic determination of the desired range of movement. A preferred development of the present invention thus provides for the setting device to be designed for at least partially automatically determining the desired range of movement by evaluating user-defined default data and/or historical data detected with the detection device and describing the joint movement of the joint of the person in a previous time period. User-defined default data can, for example, describe the state of health and/or the therapy progress and/or a desired type and/or speed of treatment. In particular, the default data can also contain a treatment plan in which different desired ranges of movement are assigned to different time periods. The time periods do not have to be absolutely defined once the progress of therapy, for example by evaluating detection data of the detection device, can also be tracked. For example, when the historical data describing a joint movement of the joint of the person in a previous time period, which are detected as detection data by the detection device, indicate that the desired range of movement provided so far is sufficiently well utilized, it is possible to move on to a new time period with an extended desired range of movement. In other words, it can thus be said that the desired range of movement that is currently to be used can be automatically adapted depending on the behavior of the person and/or their therapy progress.

In preferred embodiments of the present invention, the support unit, when determining support information to be output, can also be designed to use the current state of movement described by detection data of the detection device and/or the immediately preceding movement history of the joint. Put differently, and partly more specifically, a movement phase, in particular an approaching of the state of movement to an extreme position, and/or a movement history of the joint, can be taken into account by the support unit for determining the support information and/or an output time for the support information. In the case of the support information, which should lead to a better utilization of the desired range of movement, the point in time of the output or the current state of movement at the time of the output is to be evaluated as relatively important. Considering, for example, a knee movement, it is expedient to encourage the person to stretch the leg further if the leg is currently being stretched anyway. Accordingly, in the case of flection, a different, distinguishable support may be particularly expedient and easily attributable by the person if the support information occurs during deceleration of the flection process toward the corresponding inflection point.

In the context of the present invention, it is therefore particularly expedient to evaluate the current state of movement and/or the immediately preceding movement history of the joint to determine or predict the achievement of an inflection point and/or stopping point of the movement. Finally, it can generally be said that a support tailored to the current state of movement, in particular a current movement phase, takes place which can be thus understood intuitively by the person. In the case of a knee and/or another single-axis joint as a joint, the angular velocity can be also, in particular, considered for this purpose, thus the support information can be determined depending on the angular velocity. Thus, as already explained, the movement becomes slower shortly before turning around, i.e. shortly before the inflection point, which is an indication that the inflection point is being approached. Only then can support information for further pivoting be provided, for example. Other movement phases, for example in the case of the knee joint, can of course also be determined and taken into account within the scope of the support, for example a swinging phase and/or a push off phase.

It may be provided for the support unit to be designed to determine the support information by assessing the comparison result over a predetermined and/or predeterminable time period, in particular over a time period described by a time parameter input by means of the setting device, in particular defined as a number of movement cycles of the joint. Within the scope of the present invention, it is therefore possible to consider a time period which may be set by the user in order to assess the utilization of the desired range of movement over such time period and to decide, based on such assessment, which type of support is the most expedient. For example, an analysis can thus take place over several cycles in order to then determine, for example, a mean usage degree of the desired range of movement and/or repeatedly occurring “usage errors”/usage defects and to thus provide the support information with a sound basis. Here, it may be advantageous in particular to consider a rolling time period, thus continuously keeping the evaluation results current. A robust, sound determination of the required support and a corresponding reliable design of support information is thus possible without foregoing reasonable timeliness.

In general, in the context of the present invention it can be provided in a particularly preferred embodiment that the evaluation device is designed to control the output device for outputting the at least one piece of support information during and/or after an assessed movement. The output of the support information after an assessed movement is particularly advantageously performed directly after the same. The assessment can relate to the aforementioned time period, it being particularly expedient to realize the aforementioned “rolling time period” in order to thus be able to constantly react promptly to recent changes, in particular any progress. While the person thus uses the joint, they will at least relatively directly receive feedback on the utilization of the desired range of movement and during the movement they will receive in particular directly implementable information on how the desired range of movement can be better utilized, in the form of the support information. In the example of the knee movement, although an assessment can take place, for example, during running over several movement cycles, for example one to four movement cycles, the corresponding result can be output during the ongoing movement, in particular at suitable points in time during a movement cycle. If, for example, it is found during the assessed time period that the desired range of movement is utilized significantly more poorly during extension than during flection, current support information can be output when approaching the full extension of the knee, which will lead the person to further extend the leg, while when approaching the maximum flection, the support information can be of lower intensity, indicating that although there is still room for improvement here, the smaller deviation is present, so that the person intuitively understands that although there is still room for improvement also as regards flection, extension is the crucial issue on which the person can then particularly focus, for example, at points in time highlighted by the support information.

However, such targeted support can take place even if the evaluation does not necessarily take place over a period of time. For example, during flection it can be predicted up to where flection will take place, for example based on an occurring reduction in angular velocity, in order to then check how close this predicted angle is to the limit of the desired range of movement in order to thus proactively output support information that will lead the person to perform further flection if there is a, in particular relevant, difference to the limitation of the desired range of movement. Such an embodiment thus leads to a support that is tailored to movement phases and is more dedicated but possibly less robust or subject to stronger fluctuations than an assessment over a period of multiple movement cycles of the joint.

The output device can have a haptic and/or acoustic and/or optical output means. Where appropriate, however, other output possibilities are also conceivable, for example an olfactory output means or the like. Preferred embodiments use the output of the support information as an optical and/or acoustic, parameterizable output signal and/or also as a haptic signal, which will be discussed in more detail below.

A specific, particularly advantageous embodiment of the present invention provides for the support unit to be designed for selecting at least one output parameter, in particular the intensity and/or volume and/or frequency of the repetition of a sound and/or of a light signal, at least one of the at least one piece of support information as a function of a usage measure, determined from the comparison result, of the desired range of movement and/or approach measure to a limit of the desired range of movement. The piece of support information can in particular be output as a pulsating or basically periodic signal, wherein, for example, the period and/or the intensity can be adjusted as a function of the comparison result. In the case of an acoustic signal, it can be provided, for example, similarly to a parking assistant or the like, that a repetition frequency of a sound is increased when approaching the limit of the desired range of movement and/or that reaching the limit is acknowledged with a continuous sound. A reverse design (reduction of the repetition frequency in case of a greater approach to the limit of the desired range of movement) is also conceivable within the scope of the present invention if this has a more intuitive effect for a specific group of people. Additionally or alternatively, intensity can also increase, for example the volume, the more poorly the desired range of movement is utilized. Another specific embodiment of the present invention may also provide, for example, a plurality of individual light sources in the form of a scale as an optical output means, for example close to the joint, in particular on at least one of the parts and/or a treatment device. The better the desired range of movement is utilized, the more light sources of the scale can be brightened, for example. In this context, it is also conceivable to increasingly switch on or off output means on the joint that output the support information by vibration.

A particularly advantageous embodiment, which may be in particular used additionally, or alternatively, to an optical and/or acoustic output of the support information, provides for the medical appliance to also comprise, in particular as part of the output device, at least one stimulation device controllable by the evaluation device as a function of the comparison result for at least one muscle of the person assigned to the joint and/or at least one electrical and/or mechanical drive means designed to change the relative position of the parts and controllable by the evaluation device as a function of the comparison result. Such a stimulation device can, for example, comprise a vibration pad, which can be integrated in particular into a treatment device. Such a vibration pad is positioned such as to specifically stimulate relevant muscle groups, the activation of which results in an improved utilization of the desired range of movement in the current state of movement. If, for example, the approaching of an extreme position of a movement cycle decelerates too early, a continuation of the movement can be achieved or prompted in a targeted manner by having the stimulation device stimulate muscles. Thus, an extremely targeted piece of haptic support information can be generated.

However, it is also conceivable to selectively use an electrical and/or mechanical drive means. In this way, a clear piece of haptic support information can also be given in order, for example, to continue a movement phase even further than originally planned, for example by slightly increasing an angular velocity by operating the drive means and correspondingly influencing the relative position of the parts, in particular by keeping within limits such that a sufficiently wide movement is not “imposed” but rather at least mainly a reference function is fulfilled. For example, the effect of such a piece of haptic support information can be reduced to a maximum of 1 to 3° during a pivoting movement. Furthermore, it is also possible to use a purely mechanical drive means for outputting a piece of haptic support information. In this case, for example, the release of a spring supporting a certain movement direction can be considered, which means that the drive means can have a spring mechanically supporting a movement direction. In order to output the support information, the support unit can, for example, release and/or lock the spring.

At this point, it should be noted that the support information, which is preferably output during the movement of the joint, is in any case preferably designed such that the movement sequence is not interrupted and/or disturbed but is supported, at best.

A particularly advantageous development of the present invention provides for the evaluation device to have a classification unit that categorizes the current movement into a class of movement types based on the detection data of the detection device, the support information being output only if a class of movement types corresponding to the associated movement type is present. For example, historical data, in particular of the last movement cycles, can be evaluated in order to be able to recognize the type of movement, for example with regard to a knee or the spine as joint or joints, to recognize whether the person is currently driving a motor vehicle, climbing stairs, walking on a flat surface and the like. In particular, the support information can be limited for example to certain classes of movement types, but it is of course also possible to differentiate between classes of movement types such that, for example, different classes of movement types are associated with different desired ranges of movement and/or a different type of determination of the support information and/or different support information. For example, it may be provided that in the event of a selection of at least one desired range of movement associated with a type of movement by means of the setting device, the evaluation device is designed, in particular by means of the classification unit, to use the desired range of movement for comparison only when there is a class of movement type corresponding to the associated type of movement. In the differentiation between different classes of movement types it is thus, in particular, possible to specifically adapt support information to certain types of movement in order to thus be able to optimally work towards achieving the therapy result especially during a therapy phase.

A development of the present invention furthermore provides for the medical appliance to have a limiting device for limiting a range of movement of the joint to a permissible range of movement. Such limiting devices that are often provided, for example, as a medical appliance or treatment device in ortheses, thus make it possible to define not only a desired range of movement but also a maximum permitted range of movement, i.e. a permissible range of movement. In this context, it is particularly advantageous if the setting device is designed for adopting the permissible range of movement set by the limiting device as the desired range of movement. The desired range of movement can thus be particularly easily defined as the permissible range of movement if it is to be utilized to the maximum extent.

In addition to the medical appliance, the present invention also relates to a method for operating a medical appliance, in particular a medical appliance of the type according to the invention, for a joint of a person, comprising at least two parts which are to be attached to the joint and which are movable relative to one another by the joint and at least one detection device for detecting a relative movement of the parts, the method comprising the following steps:
determining, from detection data of the detection device, a range of movement of the joint, which range of movement is actually used when the treatment device is worn,
comparing the range of movement used with a desired range of movement,
determining support information guiding the person to the most extensive use possible of the desired range of movement, said support information being dependent on the result of the comparison, and
outputting the support information to the person.

All statements relating to the medical appliance according to the invention can be analogously applied to the method according to the invention so as to obtain the aforementioned advantages therewith.

In particular, it can also be provided according to the method of the invention that the desired range of movement to be used by the person when wearing the appliance is set at a setting device, in particular based on a user input.

Further advantages and details of the present invention result from the exemplary embodiments described below and from the drawing. The following is shown:

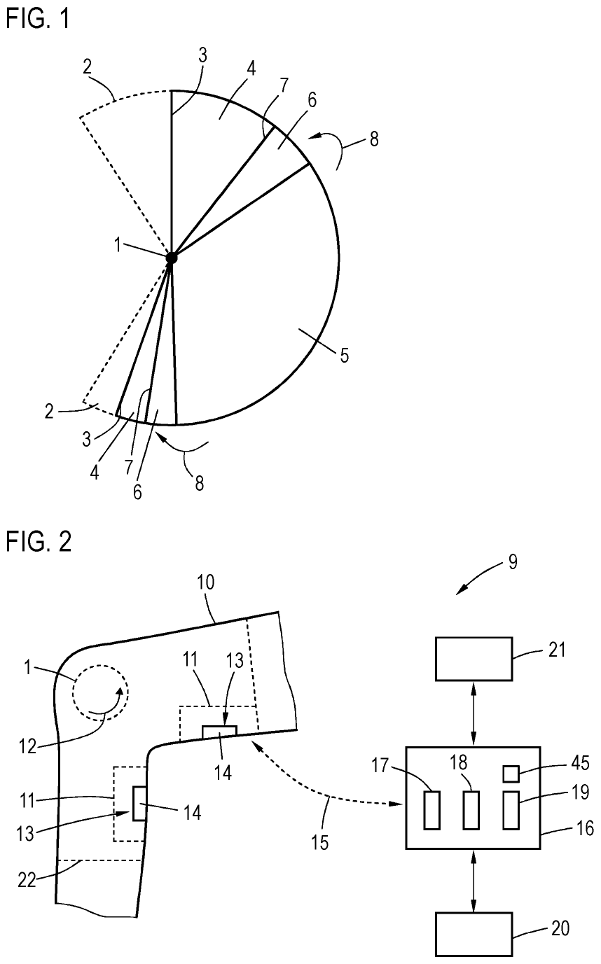

FIG. 1 shows a diagram for explaining the problem and approach underlying the invention,

FIG. 2 shows a schematic diagram of components of a medical implement according to the invention,

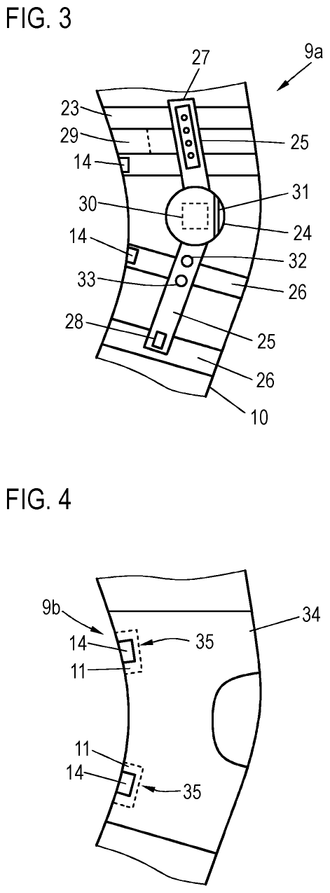

FIG. 3 shows an orthosis as an exemplary embodiment of a medical implement according to the invention,

FIG. 4 shows the application of a medical appliance of the invention in a bandage,

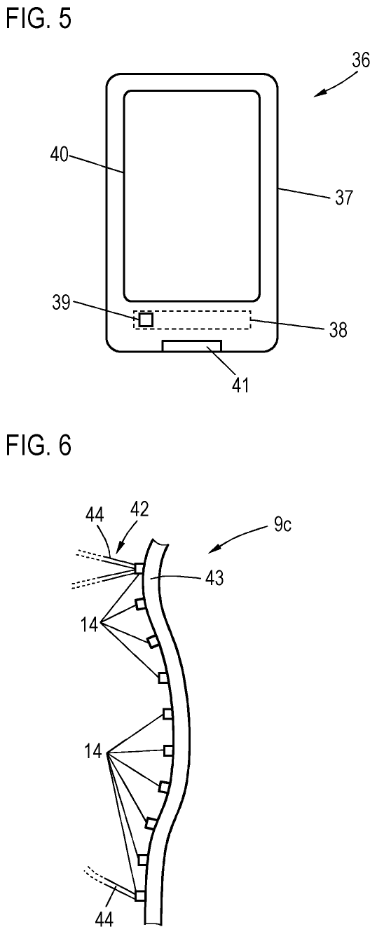

FIG. 5 shows a hand-held mobile device,

FIG. 6 shows the application of the present invention in a back orthosis,

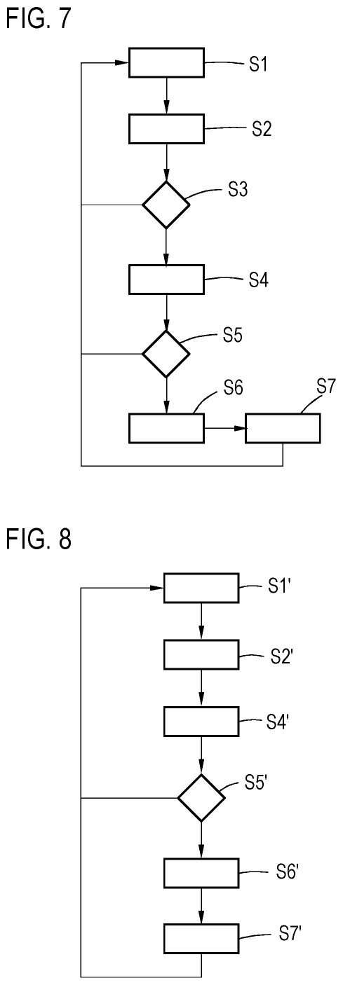

FIG. 7 shows a flow chart of a first exemplary embodiment of the method according to the invention,

FIG. 8 shows a flow chart of a second exemplary embodiment of the method according to the invention, and

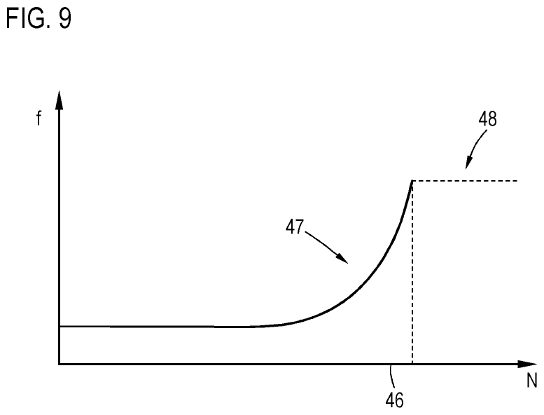

FIG. 9 shows a dependence of a repetition frequency of a signal on the degree of usage.

FIG. 1 explains the underlying idea of the present invention based on various ranges of motion (ROM) of a joint 1 of a person, such as a knee joint, as indicated by the central point. In the present case, the maximum range of movement 2 of the joint 1 indicated by dashed lines is limited by a limiting device of a treatment device used, in particular an orthosis, so that a permissible range of movement 4 delimited by lines 3 results. Due to protective postures and protective movements during the therapy phase, patients or, in general, persons using the aforementioned treatment device use only a small part of the permissible range of movement 4, shown here as the range of movement 5 actually used when wearing the treatment device.

Furthermore, however, there is also a desired range of movement 6 which is therapeutically useful and which can be described by the limits 7. The basic idea of the present invention is now to design a medical appliance that can be assigned to or can comprise said treatment device such that the patient is specifically guided, based on support information symbolized by arrows 8, to use larger portions of the desired range of movement 6, starting from the currently used range of movement 5.

FIG. 2 shows a schematic diagram of such a medical appliance 9 according to the invention for a joint 1, here for example a knee joint or other single-axis joint, of a person 10 that has only been indicated. Parts 11 are evidently provided on the body of the person 10 adjacent to the joint 1 and are moved relative to one another by the movement of the joint 1 indicated by the arrow. A detection device 13 only indicated here, which may comprise, for example, two sensors 14 or parts of a sensor 14 that are designed to measure the relative position and/or relative orientation between them, measures the movement of the joint 1 actually taking place. Its detection data can be forwarded to an evaluation device 16 via a communication link 15 only indicated here. The evaluation device 16 comprises, first of all, a determination unit 17 that determines, from the detection data of the detection device 13, the range of movement 5 of the joint 1 actually used when wearing the medical appliance 9, in the case of a single-axis joint, as shown here for example, in particular by tracking the joint angle and determining the maximum angles. It should be noted already at this point that the detection device 13 also describes dynamic parameters in its detection data, i.e. an angular velocity in the present example.

A comparison unit 18 of the evaluation device 16 compares the range of movement 5 actually used with the desired range of movement 6. For example, the corresponding, in particular maximum joint angles describing the range of movement 5 used can be compared with the maximum and minimum joint angles describing the limits 7. Ultimately, at least one usage measure results which indicates the extent to which the desired range of movement 6 is actually being used, in particular in relation to each individual limit 7.

Finally, the evaluation device 16 also has a support unit 19 which determines a piece of support information as a function of the comparison result. The support information is thereby determined in such a way as to guide or lead the person 10 to the most extensive use possible of the desired range of movement 6, which will be explained in more detail below with regard to specific exemplary embodiments.

Support information does not have to be output at each point in time or in each situation, since at least one information criterion can be provided within the support unit, which evaluates at least the comparison result and upon the fulfillment of which a piece of or the support information is output, possibly after adequate parameterization. Furthermore, it can be generally said that, when it comes to guiding the person 10 to the most extensive use possible of the desired range of movement 6, it is expedient to take into account the current state of movement and at least the preceding movement history also in the design of the support information, especially during its specific parameterization, so that the support information contains an instruction that can be intuitively understood by the person 10, namely how to achieve a broader use of the desired range of movement 6.

The support information determined by the support unit 19 can be output via a corresponding output device 20. It may comprise, for example, corresponding output means for optical and/or acoustic and/or haptic output of the support information, as will be discussed in more detail in the specific example.

In preferred exemplary embodiments, the medical appliance 9 according to the invention furthermore also has an input device 21 with at least one input means via which the desired range of movement 6 may be defined manually and/or automatically, wherein it is preferred to take a user input as a basis. An input means can be provided in particular also in mechanical form at the parts 11 and/or a treatment device 22, which is shown in FIG. 2 and which may also belong to the medical appliance 9, as a mechanical input means, for example in the case of the limiting device already addressed with regard to FIG. 1, via which a desired range of movement 6 can also be defined mechanically. With regard to automatic determination, for example, a dynamic or defined therapy plan may be preset for the person 10 by a therapist as user, which, for example, extends the desired range of movement 6 in successive periods of time for different therapy progresses. The therapy progress may follow a defined chronological sequence; however, it is preferred to track the therapy progress via the detection data since, in particular, the aforementioned usage measure determined by the comparison unit 18 offers an excellent indication as it indicates the extent to which a desired range of movement 6 has already been used, so that this may optionally be considered in order to be able to progress to a next therapy level and thus to an extended desired range of movement 6.

In addition, such a therapy plan can also entail the use of different treatment devices 22 since, for example, first an orthosis, then a bandage, and finally a belt system or the like may be used. Since the parts and/or the at least one sensor are preferably releasably attachable to the treatment device 22, which may also include the parts 11, for example via hook-and-loop fasteners, which may also apply to other components of the medical implement 9, the medical implement 9 may thus also be used with different treatment devices 22 in certain embodiments.

Generally, it should also be noted at this point that the parts 11 may, but do not have to belong to the treatment device 22. If the treatment device 22 is an orthosis for example, the parts 11 may be rigid articulated guide elements which are coupled in an articulated manner; but it is also possible for the parts 11 to be formed by subregions of a flexible component of the treatment device 22 or medical appliance 9, for example made of compression material in the case of a bandage.

Furthermore, initially it also applies in general that the evaluation device 16, the output device 20 and the setting device 21 may be implemented at least partially on the treatment device 22, i.e. close to the joint, and/or at least partially external to the treatment device 22, in particular remote from the joint 1, wherein the communication link 15 is then preferably a wireless communication link. Specific embodiments will be explained in more detail below.

As was described, the sensor 14 preferably measures the relative position and orientation to the other sensor 14 or, in particular, the passive sensor component on the other part 11. Particularly preferably magnetic and/or inductive measuring principles can be used, for example the provision of magnetic material on one part and of a corresponding magnetic field sensor on the other part 11. Other conceivable embodiments include the provision of conductive material on one part 11 and of induction sensors on the other part 11.

FIG. 3 shows a first specific exemplary embodiment of a medical appliance 9a according to the invention comprising an orthosis 23 as treatment device 22, wherein only the components of the medical appliance 9a provided on the joint 1, here a knee joint, are shown in the present case. In the present case, the orthosis has two rigid articulated guide elements 25 coupled via a joint 24, which can be held on the upper leg or lower leg of the person 10 by belts 26. In the present case, the sensors 14 or sensor components of the sensor 14 are arranged on the inner belts 26 relative to the knee joint but may also be provided on the articulated guide elements 25. The sensor elements 14 that form the detection device 13 may be integrated, for example, into the belts 26.

FIG. 3 also shows various possible embodiments of output means of the output device 20. On the one hand, a scale-like output means 27 less preferably having optical output elements and preferably having haptic output elements is shown, the output elements of which output means can be switched on in succession for example, as the approaching of a complete use of the desired range of movement 6 increases, in order to indicate this to the person 10. As a further preferred acoustic output means, the output device 20 according to FIG. 3 also has a speaker 28 arranged on the orthosis 23, here on one of the rigid articulated guide elements 25, via which sounds, possibly also voice outputs, can be output as support information. Preferred is an embodiment with repeated sounds, the repetition frequency and/or volume and/or pitch of which can be changed depending on how well the desired range of movement 6 is actually being used by the person 10.

A design based on the parking assistant of a motor vehicle has proven to be a particularly intuitively conceivable embodiment, where, for example, a higher repetition frequency indicates a stronger approach to the limits 7 of the desired range of movement 6. The pitch can be used to inform the person 10 for example about the limit 7, i.e., for example, the flection direction or the extension direction. In this case, it is already expediently provided to make the output dependent on a current movement phase or a current state of movement, for example to output support information related to the flection when approaching the inflection point of the movement during extension and accordingly for flection.

Further preferred optional haptic output means are shown in FIG. 3 in the form of a vibration pad 29 provided in the present case on the upper leg, via which relevant muscle groups can be stimulated, for example as haptic information to maintain a movement direction longer, and of an electric drive means 30 in the present case at the articulation 24, which within a certain scope—preferably only as information—can somewhat force the movement in one direction, for example when it decelerates, in order to output a piece of information to the person 10 that there is still further room for desired movement here.

FIG. 3 further shows a limiting device 31 on the articulation 24, via which a permissible range of movement 4 can be set. As has already been explained, the limiting device 31 can also be understood as a mechanical setting means of the setting device 21, which in the present case can be supplemented by a setting button 32, for example a SET button. For example, initially a desired range of movement 6 can be set via the limiting device 31 by using the SET button 32, whereupon it is still possible to extend the permissible range of movement 4 relative to the desired range of movement 6 by subsequent adjustment of the limiting device 31. A further provided RESET button 33 may also be present. It can be used, for example, when reapplying the orthosis 23.

FIG. 4 shows the use of a medical appliance 9b in a bandage 34 as the treatment device 22, wherein the bandage 34 does not necessarily have to belong to the medical appliance 9b here. Shown thereof in the present case, and for the sake of clarity, are only the at least one sensor 14 and sensor-side components of a fastening means 35, here of a hook-and-loop means, which form the parts 11. By means of the fastening means 35, the at least one sensor 14 and the parts 11 can be thus releasably fastened to the bandage 34 at in particular predetermined positions, wherein the further components of the appliance 9b (evaluation device 16, output device 20 and input device 21) that are provided external to the joint are not shown.

In this respect, FIG. 5 shows a possible embodiment in which a hand-held mobile device 36, here a smartphone 37 or tablet, is used at least partially as an evaluation device 16, output device 20, and setting device 21. For this purpose, the mobile device 36 has a computing device 38 comprising in particular at least one processor, on which an application 39 (app), i.e., a computer program, is provided, which implements the corresponding parts or components of a medical appliance 9, 9a, 9b.

For example, a touchscreen 40 can be used as input means of the input device 21 via the application 39 in order to enter desired ranges of movement 6 or default data to be used for automatically determining the same. Furthermore, the display of the touchscreen 40, just like a speaker 41, can also be used as an output means of the output device 20. The computing device 38 can at least partially implement the determination unit 17, the comparison unit 18 and/or the support unit 19.

FIG. 6 shows the use of a medical appliance 9c in a treatment device 22 designed as a back orthosis 42. The back orthosis 42, which is associated with the spine as a joint or articulated group, has a back splint 43, which is held by a belt system 44. In this case, a larger number of sensors 14 are arranged along the back splint 43 as articulated guide element in order to measure the movement of the spine as precisely as possible. Ranges of movement 4, 5, and 6 can be described, for example, by local limits of movement or the like.

Further components of the medical appliance 9c are again not shown for the sake of clarity; the back orthosis 42 may, but does not have to be associated with the medical appliance 9c as a treatment device 22.

It should also be noted at this point that the belt system 44, the bandage 34 and the belts 26 ultimately form enclosure sections, which may serve as parts, at least in regions, when the treatment device 22 belongs to the corresponding medical appliances 9, 9a, 9b, 9c. It should also be noted that, similarly to the bandage 34, a stocking or other garment may also be used as treatment device 22 or part of the appliance 9, 9a, 9b, 9c.

Returning to FIG. 2, the evaluation device 16 may also additionally comprise a classification unit 45 which may categorize the current movement into a class of movement types based on the detection data of the detection device 13. In this case, it can be provided in particular that support information is output, that different support information or different desired ranges of movement 6 are defined for different types of movement and the like only in the case of at least one specific type of movement. For example, it may be desired to provide other desired ranges of movement 6 or other support information for a knee joint as joint 1 when climbing stairs and walking normally on a flat surface. In this case, the detection data, in particular over a defined previous time period, can indicate sufficiently clearly which class of movement types is currently being used.

With reference to FIGS. 7 and 8, exemplary embodiments of the method according to the invention that may be performed by the described medical appliances 9, 9a, 9b, 9c will now be explained in more detail.

In the exemplary embodiment according to FIG. 7, the detection data is recorded by means of the detection device 13 in a step S1.

In a step S2, the determination unit 17 of the evaluation device 16 is used to determine from the detection data the range of movement 5 of the joint 1 actually used when wearing the treatment device, wherein further analyses of the detection data are also carried out. Namely, in the case of the exemplary embodiment in FIG. 7 not only the current state of movement, but also the immediately preceding movement history and/or dynamic parameters, for example an angular velocity in the case of a single or multi-axis joint, is taken into account. In the present case, a knee should be considered more accurately during a cyclic movement. The latter usually consists of sequences of an extension process and a flection process, wherein these two processes each alternate at inflection points which represent limits of the range of movement 5 actually used. If the state of movement approaches the inflection point, this is expressed for example by a reduction in angular velocity. However, this makes it possible to already determine before reaching the inflection point that said inflection point is being approached, in particular also which inflection point, and also to predict up to where the current movement process is expected to be performed, in particular up to which joint angle.

Based on the information determined in step S2, a check is made in a step S3 as to whether a relevant movement phase or a relevant state of movement is present at all. This can be regarded as a first information criterion, since it is ultimately checked whether intuitively detectable and therefore reasonable support information would be possible at all in the current state of movement or the current movement phase. If this is not the case, step S1 is returned to or step S4 is proceeded with. In step S4, the comparison unit 18 of the evaluation device 16 checks to what extent in the specific example the predicted inflection point reaches the corresponding limit 7 of the desired range of movement 6. In the example of the knee as a single-axis joint, an angular distance between the limit 7 and the predicted inflection point can be determined, for example. Of course, more complex analyses/usages are also conceivable in other exemplary embodiments.

In a step S5, it can be checked as part of a further information criterion whether the deviation from the limit 7 determined in step S4 even requires support information. If this is not the case, for example because the limit 7 is reached (or even exceeded) anyway, step S1 is returned to. It should be noted, however, that it is also absolutely conceivable to output affirmative support information even in the event of complete or almost complete use of the range of movement 6.

In a step S6, the support information is then determined as a function of the comparison result of step S4 in order to be output in a step S7.

In step S6, the support information is in particular parameterized as a function of the information collected from the detection data and, in particular, of the comparison result. For example, the further the angular distance from the limit 7, the more the intensity of support information that intuitively promotes a further movement towards the limit 7 is increased. If, for example, a stimulation device, such as the vibration pad 29, and/or a drive means 30 is used, a stronger stimulation/stronger support information can take place if the person 10 is to be recommended a longer continuation of a current movement process, for example extension or flection. The same may apply to acoustic and/or optical support information; however, it may also be expedient to intuitively convey an approach to the limit 7 by higher intensity.

FIG. 8 shows a second, exemplary embodiment of the method according to the invention that may be used as an alternative or in combination with FIG. 7.

There as well, the movement is measured in a step S1 by recording detection data. In a step S2, however, a consideration over a plurality of movement cycles in the present case takes place in the determination unit, which means that detection data of the past that describe the movement history in a time period immediately before the current point in time, are used so that a more robust description of the actually used range of movement 5 can result. For example, averages—in particular also weighted averages—can be formed for extreme positions, and the like. Here, a continuous update with all new detection data is carried out, which means that a rolling time period is considered in order to be able to react as quickly as possible to changes, in particular improvements or deteriorations as regards the desired range of movement 6.

In a step S4′, the comparison takes place in the comparison unit 18 since in this exemplary embodiment continuous information, for guiding or leading the person, is desired anyway by means of support information.

Nevertheless, in a step S5′, which is in turn optional, information criteria may be checked as to whether support information is required at all. Especially in the event of an ultimately desired continuous guidance of the person 10 towards an improved utilization of the desired range of movement 6, it may also be desired to no longer output any support information if there is sufficient usage.

In a step S6′, analogous to step S6 in FIG. 7, the support information is determined and output in step S7. In this exemplary embodiment, it is preferred to use a tone sequence, the repetition frequency of which depends on the degree of usage, here in particular on the approaching of the limits 7.

FIG. 9 shows such an exemplary dependence of a repetition frequency f of a tone on the degree of usage N. The value 46 here indicates a substantially complete use of the desired range of movement 6. Obviously, there is first a low repetition frequency, which increases significantly as the use in the region 47 increases and thus intuitively conveys said approaching of the limits 7 similarly to a “parking assistant”. From value 46 upwards, region 48, for example a continuous tone can be output and/or the support information can be deactivated because use of the desired range of movement 6 to the greatest degree possible has been reached.

Finally, it should be noted that it is expedient not only in the exemplary embodiments according to FIG. 7 and FIG. 8 but generally in all embodiments of the invention to intuitively output the support information in a manner assignable to the movement during the movement itself, in particular immediately after the assessment. This improves the assignment, in particular when reacting to specific movement phases or predictions, and thus also the leading and guiding effect for improved utilization of the desired range of movement 6. It should also be noted in general that the support information preferably does not interfere with or interrupt the movement itself, but intuitively flows into the movement sequences thus leading the user to an improved utilization of the desired range of movement 6.

1. Medical appliance (9, 9a, 9b, 9c) for a joint (1) of a person (10), which has at least two parts (11) which are to be placed on the joint (1) and can be moved with respect to one another by the joint (1) and at least one detection device (13) for detecting a relative movement of the parts (11), characterized by an evaluation device (16), having:
a determination unit (17) for determining, from detection data of the detection device (13), a range of movement (5) of the joint (1), which range of movement is actually used when the medical appliance (9, 9a, 9b, 9c) is worn,
a comparison unit (18) for comparing the range of movement (5) used with a desired range of movement (6), and
a support unit (19) for determining support information guiding the person (10) to the most extensive use possible of the desired range of movement (6), said support information being dependent on the result of the comparison, wherein the medical appliance (9, 9a, 9b, 9c) further has an output device (20) for outputting the support information to the person (10).

2. Medical appliance (9, 9a, 9b, 9c) according to claim 1, characterized in that the medical appliance (9, 9a, 9b, 9c) has at least one stocking-like enclosure section which is to be pulled over the joint (1) and/or wrapped around the joint (1) as a bandage and/or designed as a garment and/or as a belt system (44) and/or at least one articulated guide element (25), in particular at least two hinged, rigid articulated guide elements (25).

3. Medical appliance (9, 9a, 9b, 9c) according to claim 1, characterized in that the detection device (13) comprises at least one sensor (14) integrated into at least one part (11) or detachably or non-detachably attached or attachable to at least one part (11) and/or to a body region moving with a part (11), and/or is designed to measure a distance between two sensors (14) and/or sensor parts and/or a joint angle and/or at least one dynamic variable of the joint movement, in particular an angular velocity and/or an angular acceleration, and/or that the at least one sensor (14) has a measuring principle using magnetism and/or induction.

4. Medical appliance (9, 9a, 9b, 9c) according to claim 1, characterized in that the appliance (9, 9a, 9b, 9c) has a setting device (21) for setting the desired range of movement (6) of the joint (1) which the person (10) is to use when wearing the appliance (9, 9a, 9b, 9c).

5. Medical appliance (9, 9a, 9b, 9c) according to claim 4, characterized in that the setting device (21) has an electronic and/or mechanical setting means, in particular one that exists independently of a treatment device (22) formed by the parts (11), in particular a hand-held mobile device (36).

6. Medical appliance (9, 9a, 9b, 9c) according to claim 5, characterized in that the mobile device (36) additionally comprises a computing device (38) designed as at least one part of the evaluation device (16), on which computing device (38) an application (39) exists that configures the mobile device (36) as a setting means and as the part of the evaluation device (16).

7. Medical appliance (9, 9a, 9b, 9c) according to claim 4, characterized in that the setting device (21) is designed to at least partially automatically determine the desired range of movement (6) by evaluating user-defined default data and/or historical data detected with the detection device (13) and describing the joint movement of the joint (1) of the person (10) in a previous time period.

8. Medical appliance (9, 9a, 9b, 9c) according to claim 1, characterized in that a movement phase, in particular an approaching of the state of movement to an extreme position, and/or a movement history of the joint (1), can be taken into account by the support unit (19) for determining the support information and/or an output time for the support information.

9. Medical appliance (9, 9a, 9b, 9c) according to claim 1, characterized in that the evaluation device (16) is designed to control the output device (20) for outputting the at least one piece of support information during and/or after an assessed movement.

10. Medical appliance (9, 9a, 9b, 9c) according to claim 1, characterized in that the support unit (19) is designed for selecting at least one output parameter, in particular the intensity and/or volume and/or frequency of the repetition of a sound and/or of a light signal, at least one of the at least one piece of support information as a function of a usage measure, determined from the comparison result, of the desired range of movement (6) and/or approach measure to a limit (7) of the desired range of movement (6).

11. Medical appliance (9, 9a, 9b, 9c) according to claim 1, characterized in that it also comprises, in particular as part of the output device (20), at least one stimulation device controllable by the evaluation device (16) as a function of the comparison result for at least one muscle of the person (10) assigned to the joint (1) and/or at least one electrical and/or mechanical drive means (30) designed to change the relative position of the parts (11) and controllable by the evaluation device (16) as a function of the comparison result.

12. Medical appliance (9, 9a, 9b, 9c) according to claim 1, characterized in that the evaluation device (16) has a classification unit (45) that categorizes the current movement into a class of movement types based on the detection data of the detection device (13), the support information being output only if a class of movement types corresponding to the associated movement type is present.

13. Medical appliance (9, 9a, 9b, 9c) according to claim 1, characterized in that it comprises a limiting device (31) for limiting a range of movement (2) of the joint (1) to a permissible range of movement (4).

14. Method for operating a medical appliance (9, 9a, 9b, 9c) for a joint (1) of a person (10), comprising at least two parts (11) which are to be placed on the joint (1) and can be moved with respect to one another by the joint (1) and at least one detection device (13) for detecting a relative movement of the parts (11), the method comprising the following steps:
determining, from detection data of the detection device (13), a range of movement (5) of the joint (1), which range of movement is actually used when the treatment device (22) is worn,
comparing the range of movement (5) used with a desired range of movement (6),
determining support information guiding the person (10) to the most extensive use possible of the desired range of movement (6), said support information being dependent on the result of the comparison, and
outputting the support information to the person (10).

15. Method according to claim 14, characterized in that the desired range of movement (6) to be used by the person (10) when wearing the appliance (9, 9a, 9b, 9c) is set at a setting device (21), in particular based on a user input.
